(19) United States
(12) Plant Patent Application Publication
(10) Pub. No.: US 2003/0150033 P1
(43) Pub. Date: Aug. 7, 2003

(54) TORENIA PLANT NAMED 'SUNRENIRIREPA'

(76) Inventor: Kiyoshi Miyazaki, Hikone-shi (JP)

Correspondence Address: BURNS, DOANE, SWECKER & MATHIS, L.L.P., P.O. Box 1404, Alexandria, VA, 22313-1404, US

(21) Appl. No.: 10/066,739

(22) Filed: Feb. 6, 2002

(51) Int. Cl. A01H 5/00

(52) U.S. Cl. PLT/263

(57) ABSTRACT

Disclosed herein is a torenia plant which has large globose form flowers and single color flower petals with vivid red purple and vivid red purple floral tube. The plant has a semi-erect growth habit, medium branching and great profusion of blooms, the whole bush remaining in bloom for a considerable period of time.

### BACKGROUND OF THE VARIETY

The present invention relates to a new and distinct variety of torenia plant obtained from mutantation of ‘SUNRENIMU’ (Torenia hybrid).

The torenia is a very popular plant and is used for flower bedding and potting in the summer season. There are only a few varieties of the torenia plant which have a semi-erect growth habit, medium branching and great profusion of blooms.

Accordingly, this invention is aimed at obtaining a new variety having a great profusion of flowers, vivid red purple flower petal without yellow eye color, semi-erect growth habit, medium branching and great profusion of blooms.

The new variety of torenia plant according to this invention originated from mutation occurring as a spontaneously generic variant of ‘SUNRENIMU’ which was previously filed in Japan [‘SUNRENIMU’ The Seeds and Seedlings Law Registration No. 7181]] and United States of America [TORENIA PLANT ‘SUNRENIMU’ (U.S. Plant Pat. No. 10,120)], and so on. The new variety of torenia plant was discovered on the basis of the flower color during propagation of ‘SUNRENIMU’ in January of 1997 in the Omi Research Center of SUNTORY Ltd., located at 863-1, Aza-Iketani, Omori-cho, Yokaichi-shi, Shiga-ken, Japan. The discovered torenia plant was propagated by cutting from January of 1997 and then grown in bed and pot on trial from April 1997. The botanical characteristics of the plant were examined, using a similar variety, ‘Crown Violet’ and parent variety, ‘SUNRENIMU’, for comparison. As a result, it was concluded that this torenia plant is distinguishable from other varieties, whose existence is known, uniform and stable in its characteristics, then this new variety of torenia plant was named ‘SUNRENIRIREPA’ (Torenia hybrid).

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plant (J.H.S. Color Chart) is also added for reference.

‘SUNRENIMU’, the mutant parent variety of ‘SUNRENIRIREPA’ was obtained from crossing of ‘Crown Violet’ having purplish white flower petals as the female parent and ‘Con Color’ having strong bluish purple flower petals as the pollen parent, in February of 1993, in the Plant Biotechnology Laboratory, Institute for Fundamental Research of SUNTORY Ltd., located at 1-1, Wakayamadai 1-chome, Shimamoto-cho, Mishima-gun, Osaka-fu, Japan.

The female parent used in the breeding of ‘SUNRENIMU’ and used as the comparison variety for the new variety ‘SUNRENIRIREPA’ was ‘Crown Violet’ having purplish white flower petals, one of the Crown Series bred by the Sakata Seed Corp., Japan. The Crown Series includes plants having white flower petals, purplish blue flower petals, bi-color of white and purplish blue and the like, and these plants are commonly characterized by a high resistance to heat and are available on the market in Japan. The main botanical characteristics of ‘Crown Violet’ are as follows.

Plant:

Growth habit.—Erect.

Plant height.—20-30 cm.

Plant extension.—30-50 cm.

Stem:

Diameter.—3.0 mm.

Anthocyanin pigmentation.—Present.

Branching.—Few.

Pubescence.—Sparse.

Length of internode.—1-3 cm.

Leaf:

Phyllotaxis.—opposite.

Shape of blade.—Serrate.

Length.—2.0-4.0 cm.

Width.—2.0-3.0 cm.

Depth of incision.—Deep.

Color.—Dark yellow green (R.H.S. 137A, JHS 3508).

Pubescence of upper side.—Sparse.

Flower:

Facing direction.—Lateral.

Diameter.—2.0-3.0 cm.

Height.—20-30 mm.

Color of floral tube.—Soft violet (R.H.S. 92C, JHS 8008).

Color of petal.—Single color; purplish white (R.H.S. 62D, JHS 8001).

Yellow eye color.—Present. Vivid yellow (R.H.S. 17C, JHS 2507).

Calyx.—1.5-2.0 cm in length.

Anthocyanin pigmentation of calyx limb.—Present.

Peduncle.—1.5-2.0 mm in thickness; and 1.5-2.0 cm in length.

Reproductive organs.—1 pistil and 4 stamens.

Anther color.—White.

Flowering duration.—Medium.

Physiological and ecological characteristics: Low resistance to diseases and pests, high tolerance to heat and low tolerance to cold.

One of the species of Torenia ‘Con Color’, used as the male parent in the breeding of the variety ‘SUNRENIMU’, is one of the Con Color Series bred by the Sakata Seed Corp., Japan. The Con Color Series includes plants having violet flower petal, bi-color of strong purple and pale purple and the like, and these plants are commonly characterized by a high resistance to heat and are available on the market in Japan. The main botanical characteristics of ‘Con Color’ are as follows.

Plant:

Growth habit.—Decumbent.

Plant height.—10-15 cm.

Plant extension.—50-70 cm.

Stem:

Diameter.—1.5 mm.

Anthocyanin pigmentation.—Present.

Branching.—Many.

Pubescence.—Sparse.

Length of internode.—4-6 cm.

Leaf:

Phyllotaxis.—Opposite.

Shape of blade.—Crenate.

Length.—1.0-2.0 cm.

Width.—1.0-2.0 cm.

Depth of incision.—Medium.

Color (upper side).—Moderate olive green (R.H.S. 137A, JHS 3508).

Pubescence of upper side.—Sparse.

Flower:

Facing direction.—Lateral.

Diameter.—2.0-3.0 cm.

Height.—20-30 mm.

Color of floral tube.—Moderate purple (R.H.S. 83B, JHS 8613).

Color of petal.—Single color; Deep purple (R.H.S. 89C, JHS 8311).

Yellow eye color.—Absent.

Calyx.—1.5-2.0 cm in length.

Anthocyanin pigmentation of calyx limb.—Present.

Peduncle.—1.5-2.0 mm in thickness; and 3.0-5.0 cm in length.

Reproductive organs.—1 pistil and 4 stamens.

Anther color.—White.

Flowering duration.—Medium.

Physiological and ecological characteristics: High resistance to diseases and pests, high tolerance to heat and low tolerance to cold.

We previously filed ‘SUNRENIMU’ which is the mutant parent of the variety ‘SUNRENIRIREPA’ in Japan [‘SUNRENIMU’ The Seeds and Seedlings Law Registration No. 7181]] and the United States of America [TORENIA PLANT ‘SUNRENIMU’ (U.S. Plant Pat. No. 10,120)], and so on. The botanical characteristics of the torenia plant ‘SUNRENIMU’ is as follows:

Plant:

Growth habit.—Semi-erect. The stems hang down pliantly when potted in a hanging pot.

Plant height.—10-20 cm.

Plant extension.—50-60 cm. The stem extends to length of 60 cm from the base.

Growth.—Medium branching, a great profusion of blooms; the whole bush remaining in bloom for a considerable period of time.

Blooming period.—June to November in the southern Kanto area, Japan. The plant shape does not change throughout this period.

Stem:

Diameter.—2.0 mm.

Anthocyanin pigmentation.—Present.

Branching.—Medium.

Pubescence.—Sparse.

Length of internode.—6-8 cm.

Leaf:

Phyllotaxis.—Opposite.

Shape of blade.—Serrate.

Length.—2.0-3.0 cm.

Width.—1.5-2.5 cm.

Depth of incision.—Medium.

Color (upper side).—Moderate olive green (R.H.S. 137A, JHS 3508).

Pubescence of upper side.—Sparse.

Flower:

Facing direction.—Lateral.

Diameter.—2.0-3.0 cm.

Height.—30-40 mm.

Color of floral tube.—Light purple (R.H.S. 92B, JHS 8303).

Color of petal.—Single color; dark reddish purple (R.H.S. 83B, JHS 8909).

Yellow eye color.—Absent.

Calyx.—1.5-2.0 cm in length.

Anthocyanin pigmentation of calyx limb.—Present.

Peduncle.—1.5-2.0 mm in thickness; and 2.0-3.0 cm in length.

Reproductive organs.—1 pistil and 4 stamens.

Anther color.—White.

Flowering duration.—Medium.

Physiological and ecological characteristics: Medium resistance to diseases and pests, high tolerance to heat and low tolerance to cold. The plant grows and has flowers ordinarily in the shade of trees.

### SUMMARY OF THE VARIETY

The new variety of torenia plant named ‘SUNRENIRIREPA’ has a semi-erect habit, vivid red purple flower petal without yellow blotches, and thus is outstanding among similar varieties, ‘Crown Violet’ having purplish white single color flower petals. The new plant has a semi-erect habit, medium branching and great profusion of blooms, and the whole bush remains in bloom for a considerable period of time. The new variety ‘SUNRENIRIREPA’ has large globose form flower and vivid red purple single color flower petals without yellow eye color, which is clearly distinguished from ‘SUNRENIMU’ having large globose form flowers and dark reddish purple single color flower petals.

### BRIEF DESCRIPTION OF THE DRAWING

FIG. 1 is a photograph showing a partial view of the new variety of torenia plant planted in a flower pot;

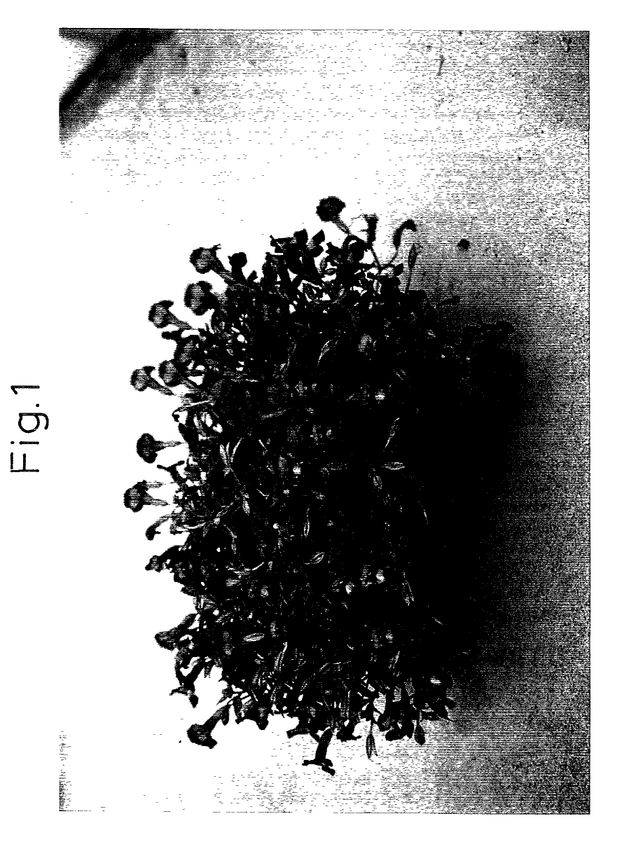

FIG. 2 is a photograph of flowers of the new variety of torenia plant.

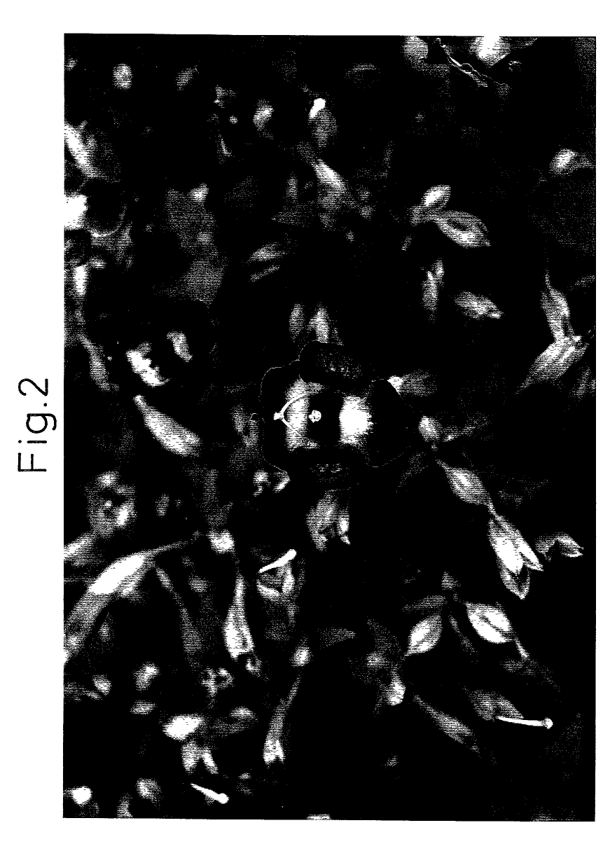

### DESCRIPTION OF THE VARIETY

The botanical characteristics of the new and distinct variety of torenia plant ‘SUNRENIRIREPA’ are as follows. This investigation was held at the end of July at Kitakoma-gun, Yamanashi-ken, Japan. Plant materials, propagated by stem cuttings, examined in this investigation had been cultivated since April to June.

Plant:

Growth habit.—Semi-erect. The stems hang down pliantly when potted in a hanging pot.

Plant height.—20 cm.

Growth.—Medium branching, a great profusion of blooms; the whole bush remaining in bloom for a considerable period of time.

Blooming period.—June to November in the southern Kanto area, Japan. The plant shape does not change throughout this period.

Stem:

Diameter.—2.5 mm.

Anthocyanin pigmentation.—Present. Medium.

Branching.—Medium.

Pubescence.—Sparse.

Length of internode.—3.1 cm.

Leaf:

Phyllotaxis.—Opposite.

Shape of blade.—Lanceolate.

Length.—2.9 cm.

Width.—1.9 cm.

Depth of incision.—Medium.

Color (upper side).—Moderate olive green (R.H.S. 137A, JHS 3508).

Pubescence of upper side.—Sparse.

Thickness of petiole.—Thick.(2.2 mm).

Length of petiole.—Short.(0.5 cm).

Flower:

Inflorescence peduncles.—Axillary.

Flower form.—Globose.

Flower length.—Medium. (28 mm).

Flower width.—Medium. (26 mm).

Length of tubular.—Short. (22 mm).

Color of floral tube.—Strong reddish purple. (R.H.S. 78A, JHS 9208).

Color of petal.—Single color. Strong reddish purple. (R.H.S. 78A, JHS 9208).

Yellow petal blotches.—Absent.

Vertical petal lines.—Present.

Upper bilabiate petal wave.—Strong.

Calyx shape.—Shallowly five-lobed.

Calyx;Degree of development of wings.—Medium.

Calyx length.—Short to medium. (15 mm).

Anthocyanin pigmentation of calyx limb.—Present. Dark.

Anthocyanin pigmentation of anther.—Present. Medium.

Anther spur.—Absent.

Anther color.—White.

Peduncle thickness.—Medium. (1.7 mm).

Peduncle length.—Short to medium. (22 mm).

Cluster.—Absent.

Number of flowers on each stem.—Few.

Reproductive organs.—1 pistil and 4 stamens.

Flowering duration.—Medium.

Physiological and ecological characteristics: Medium resistance to heat, cold, diseases and pests.

This new variety ‘SUNRENIRIREPA’ of torenia plant is most suitable for flower bedding and potting, particularly in hanging pots or planters.

This new variety ‘SUNRENIRIREPA’ was asexually reproduced by cutting.

The plant of this new variety ‘SUNRENIRIREPA’ is presently planted and maintained in the Omi Research Center of SUNTORY Ltd.

I claim:

1. A new and distinct variety of torenia plant, substantially as herein illustrated and described, characterized particularly as to novelty by (A) having large globose form flowers and single-color vivid red purple flower petals without yellow blotches, and vivid red purple floral tube, (B) a semi-erect growth habit, medium branching and great profusion of blooms.
